(19) United States
(12) Patent Application Publication
(10) Pub. No.: US 2020/0377620 A1
(43) Pub. Date: Dec. 3, 2020

(54) CLOSTRIDIUM PERFRINGENS SURFACE GLYCANS AND USES THEREOF

(71) Applicant: THE GOVERNORS OF THE UNIVERSITY OF ALBERTA, Edmonton (CA)

(72) Inventors: Christine SZYMANSKI, Edmonton, AB (CA); Cory WENZEL, Edmonton, AB (CA); Mario FELDMAN, Edmonton, AB (CA); Dominic MILLS, Edmonton, AB (CA)

(21) Appl. No.: 16/770,275

(22) PCT Filed: Dec. 19, 2018
(86) PCT No.: PCT/CA2018/051627

§ 371 Date: Jun. 5, 2020

Related U.S. Application Data
(60) Provisional application No. 62/607,848, filed on Dec. 19, 2017.

(51) Int. Cl. C08B 37/00; A61K 39/08; A61P 31/04; G01N 33/569; A61P 37/04

(52) U.S. Cl. CPC C08B 37/006; A61K 39/08; A61K 2039/6018; G01N 33/56911; A61P 37/04; A61P 31/04

(57) ABSTRACT

An immunogenic glycan compound has a poly-β-1,4-ManNAc repeating-unit structure variably modified with 6-linked phosphoethanolamine and 6-linked phosphoglycerol.

### FIELD OF THE INVENTION

The present application pertains to Clostridium perfringens surface glycans and uses thereof in vaccines and in the diagnosis and treatment of infections caused by C. perfringens.

### BACKGROUND

Clostridium perfringens is a Gram-positive toxin-producing anaerobic bacterium that is one of the most common causes of foodborne illness in humans (Grass et al. (2013)), and is also responsible for enteric diseases in numerous species of livestock (Sanger (1996); Uzal et al. (2010)). C. perfringens is the primary cause of avian necrotic enteritis (NE) (Al-Sheikhly et al. (1977a); Timbermont et al. (2010)), which poses a significant problem in the poultry industry. The disease leads to rapid death within 24 hours of the onset of acute infection, precluding treatment in most cases (Caly et al. (2015), and subclinical infections are associated with chronic damage to the intestinal mucosa, leading to reduced weight gain and lower feed efficiency (Elwinger et al. (1998); Hotkre et al. (2003); Hofshagen et al. (1992); Kaldhusdal et al. (2001)). Combined. NE is estimated to be responsible for S2 billion dollars in annual losses worldwide for the poultry industry (Van der Sluis (2000)). Furthermore, the European ban on the prophylactic use of antibiotics with livestock (European-Union, Regulation (EC) No 1831/2003) has resulted in an increase in NE outbreaks in European countries (Van Immersed. et al. (2004)) that has led to a 33% loss in profit for flocks heavily infected with C. perfringens compared to healthy flocks (Lovland et al. (2001)). These losses highlight the need for alternative prevention strategies in place of antibiotic therapy.

Despite the importance of C. perfringens in a livestock context and the identification of capsular polysaccharide (CPS) as the primary antigenic determinant of the Hobbs typing scheme (Hughes et al. (1976)), little research has been done to identify and characterize carbohydrate structures present on the surface of this organism. Only the CPS structures from C. perfringens Hobbs 5, 9, and 10 have been examined in any detail, whereby the composition of the Hobbs 9 CPS was determined to be glucose (G1c), galactose (Ga1) and galactosamine (Ga1N) in a 1:1.6:1.1 ratio in 1977 (Cherniak et al. (1977)), and the complete structures of the Hobbs 5 and Hobbs 10 CPS were solved by NMR spectroscopy in 1997 and 1998, respectively (Kalelkar et al. (1997); and Sheng et al. (1997)).

In addition to CPS structures, many Gram-positive bacteria produce cell wall teichoic acids (WTA) and lipoteichoic acids (LTA), but little has been done to examine for the presence and potential importance of these or other carbohydrate structures in C. perfringens. Richter et al (2013) noted the presence of three homologues of the LTA synthase gene (ItaS) in the genome of C. perfringens SM101, and demonstrated that C. perfringens SM101 was very sensitive to a small molecule inhibitor of LTA synthesis, suggesting the presence and importance of LTA in C. perfringens, yet the presence of LTA has not been demonstrated nor structurally characterized in this bacterium until very recently, when Vinogradov et al. (2017) reported that C. perfringens ATCC 13124 produces an LTA with a repeating structure of β-ManNAc6)PEtN-(1→4)-[β-ManNAc6PEtN-(1→4)]-β-ManNAc-(1→4)-β-ManNAc6PEtN [3-Ribf]-(1→4)-β-ManN-(1→4)-β-G1c-(1→1)-Gro.

There are no known polysaccharide-based vaccines against C. perfringens. Vaccination strategies to-date have centred on the use of protein antigens, such as detoxified versions of toxins produced by C. perfringens (toxoid) and C. perfringens surface and secreted proteins, resulting in varying degrees of protection (Mot et al. (2014)). Due to the production of more than one toxin by C. perfringens strains causing livestock diseases, including NE in chickens, effective protein vaccine strategies may require multi-valent vaccines containing more than one toxoid.

Commercially available C. perfringens vaccines for poultry (Netvax®(Merck Animal Health, Whitehouse Station, N.J.) and Clostridium Toxoid Autovaccine (Vacci-Vet™, Saint-Hyacinthe, QC, Canada), are based on alpha-toxin toxoids, but the toxin NetB has since been shown to play a more pivotal role in C. perfringens pathology in chickens. Moreover, a recent NE vaccine study found that significant protection levels were only observed when a combination of alpha toxin- and NetB-derived antigens were used (Jiang et al. (2015)). One of the major considerations in the development of an NE vaccine is that it must be inexpensive to produce due to the low market value of chickens, and vaccine strategies requiring multiple antigens rather than a single antigen may prove t be cost prohibitive for use in poultry.

There remains a need to identify a conserved, immunogenic target molecule from C. perfringens that elicits a widely cross-reactive immune response to be used as the primary antigen in a safe and effective vaccine against NE in chickens, other livestock diseases, and human food-poisoning caused by C. perfringens.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

The present invention is based on the identification of a conserved C. perfringens antigen that comprises a polysaccharide with a poly-β-1,4-ManNAc repeating-unit structure variably modified with 6-linked phosphoethanol amine and 6-linked phosphoglycerol. In general terms, the invention comprises an immunogenic glycan compound comprising a poly-β-1,4-ManNAc repeating-unit structure, modified with at least one 6-linked phosphoglycerol.

In one aspect, the invention may comprise an immunogenic Clostridium perfringens-specific surface glycan, which comprises the compound of Formula I, in isolated, synthesized and/or purified form, lipid-linked or free or an analogue or modified form thereof:

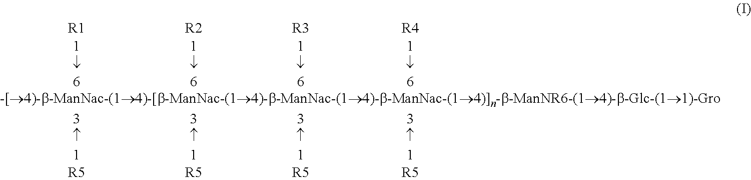

where n≥1, G1c represents glucose, ManNAc represents N-acetylmannosamine (2-acetamido-2,6-dideoxy-mannose), ManN represents mannosamine (2-amino-2-deoxy-D-mannopyranose), Gro represents glycerol, and where each of R1, R2, R3, and R4 comprises any substituent or modification, provided at least one of R1-R4 is phosphoglycerol (—PGro); R5 comprises any modification such as —OH; and R6 comprises —H or —Ac. In one embodiment, one R5 in a terminal copy of the repeating structure may comprise a sugar, such as Ribf (ribofuranose).

In some embodiments, the glycan of Formula I comprises a compound where at least one of R1-R4 is PGro, and at !east one, two or three of R1-R4 is phosphoethanolamine or OH.

In some embodiments, the glycan has the structure of Formula II, in isolated, synthesized and/or purified form, lipid-linked or free, or an analogue or modified form thereof:

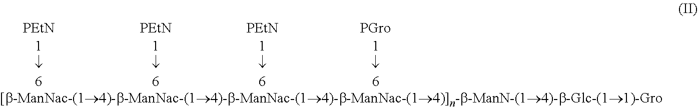

In some embodiments, a compound of Formula I or II, or an immunogenic analogue or modified form thereof, may be linked to a lipid or conjugated to a single amino acid, an oligopeptide, a peptide or a protein, for example.

In another aspect, the invention may comprise a method of producing an antibody or antiserum comprising the steps of providing a compound bearing an antigenic surface structure comprising all or a part of a glycan of Formula I or II, inoculating an animal with the compound to stimulate an immune response to the compound, withdrawing serum from said animal and optionally purifying said serum to obtain the antibody or antiserum which specifically binds to the glycan. The antibody or antiserum may be used for diagnostic purposes, to detect the presence of C. perfringens in an animal or in a human, or in a passive immunization method, to treat an actual or potential C. perfringens infection.

Compounds of the present invention may be used in a vaccine formulation, with or without an adjuvant, against C. perfringens, which vaccine formulation may be administered to poultry, such as chickens, or other livestock. The compounds may also be used in a vaccine formulation for mammals, such as humans, since C. perfringens is also a major cause of human food-poisoning from the consumption of contaminated foods, such as beef or poultry. Compounds of the present invention may also have uses in glycoconjugate vaccines and diagnostic applications.

In another aspect, the invention may comprise a vaccine which comprises an antigenic compound comprising all or part of a glycan of Formula I or II, or an analogue r modified form thereof, optionally linked to a single amino acid, an oligopeptide, a peptide, a protein, or a lipid, r borne on an attenuated C. perfringens cell or expressed on a bacteria engineered to hetcrologously express the antigenic compound.

In other aspects, the invention may comprise methods of treating or preventing an infection caused by a C. perfringens organism using a composition comprising all or part of a compound of Formula I or II, or an immunogenic analogue or modified form thereof, within a human or animal. A vaccine in accordance with the present invention may be used for improving the productivity and health of an animal by administering said vaccine as described above. Vaccines, antibodies and antisera described herein may also be used for prevention, treatment and diagnosis in subjects including humans.

### BRIEF DESCRIPTION OF THE DRAWINGS

In the drawings shown in the specification, like elements may be assigned like reference numerals. The drawings are not necessarily to scale, with the emphasis instead placed upon the principles of the present invention. Additionally, each of the embodiments depicted are but one of a number of possible arrangements utilizing the fundamental concepts of the present invention.

FIG. 1 is a Western immunoblot illustrating that the immunodominant antigen on the surface of C. perfringens is proteinase K-resistant.

FIG. 2 is a Western immunoblot illustrating that the immunodominant surface antigen of C. perfringens is a polysaccharide or glycolipid.

FIG. 3 shows Western immunoblots illustrating that the common surface polysaccharide is immunodominant in both rabbits and chickens, and that the immune response to the surface polysaccharide from C. perfringens HN13 is cross-reactive with all field isolates tested, while antiserum against the surface polysaccharide from C. perfringens JGS4143 (is only cross-reactive with a small number of field isolates. , and that the chicken anti-HN13 antiserum is dramatically less cross-reactive with the field isolates after being adsorbed against whole cells of the C. perfringens HN13 cpe2237 mutant (putative phosphoglycerol-minus mutant, isolate #3).

FIG. 4 is a Western immunoblot illustrating that the immunodominant surface antigen is not present in other Clostridium species.

FIG. 5 shows the percent survival of leghorn chicks orally gavaged with either PBS, 1×10⁹ C. perfringens JGS4143 cells in PBS, or co-gavaged with 1×10⁹ C. perfringens JGS4143 cells in 1:100 anti-C. perfringens serum:PBS.

FIG. 6 shows the percent survival of C. perfringens JGS4143 cells in an opsonophagocytosis assay evaluating the protection potential of chicken antiserum raised against whole cells of C. perfringens HN13 vs naïve chicken serum.

FIG. 7 is a Western immunoblot illustrating extracted and isolated C. perfringens immunodominant antigen from strain HN13 and chicken NE strain JGS4143.

FIG. 8 shows NMR spectroscopy data of the deacylated conserved immunodominant antigen from C. perfringens HN 13, confirming the presence of a polysaccharide with a tetrasaccharide repeating-unit structure modified with phosphoethanolamine and phosphoglycerol of Formula 11.

FIG. 9 shows NMR spectroscopy data of A) high-molecular-weight and B) low-molecular-weight forms of the deacylated and dephosphorylated conserved immunodominant antigen from C. perfringens HN13, confirming a terminal disaccharide-glycerol at the reducing end of the tetrasaccharide repeat of Formula II.

FIG. 10 shows NMR spectroscopy data of the delipidated conserved immunodominant antigen from C. perfringens JGS4143, confirming the presence of a polysaccharide consisting of a poly-ManNAc repeating-unit structure modified with phosphoethanolamine, capped at the non-reducing end with a trisaccharide modified with PEtN and at the reducing end with a disaccharide-glycerol of Formula III.

FIG. 11 shows a Western immunoblot demonstrating that the C. perfringens HN13 cpe2237 mutant, which putatively lacks phosphoglycerol, is markedly less immunoreactive against/to the chicken anti-HN13 antiserum, and that complementation of the mutant with a copy of the cpe2237 gene in trans restores the reactivity of the mutant to wildtype levels, as shown for three distinct isolates of the mutant.

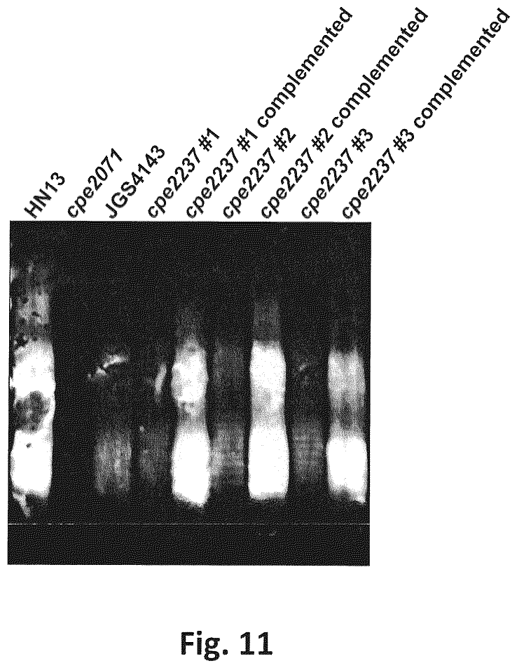

FIG. 12 shows the novel repeating-unit structure of the polysaccharide regions of the C. perfringens broadly cross-reactive common surface polysaccharide antigen described in Formula 1, as well as the broadly-cross-reactive surface polysaccharide from C. perfringens HN13 (Formula II).

FIG. 13 shows the polysaccharide region of the polysaccharide antigen from JGS4143

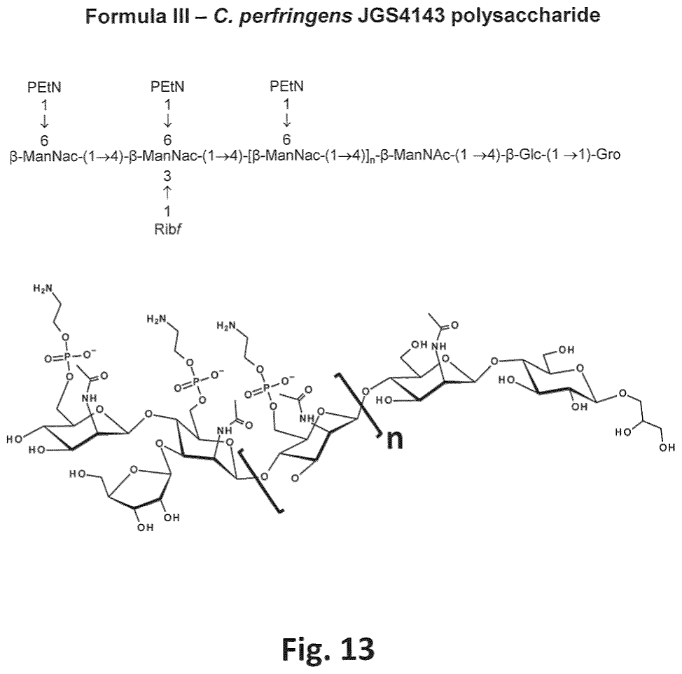

(Formula Ill) which is recognized by anti-HN13 (Formula II) antiserum but does not elicit a broadly cross-reactive immune response.

### DETAILED DESCRIPTION OF PREFERRED EMBODIMENT

Any term or expression not expressly defined herein shall have its commonly accepted definition understood by a person skilled in the art.

As used herein, a “glycan” is a polysaccharide or oligosaccharide compound consisting of a plurality of monosaccharides linked glycosidically, or is the polysaccharide or oligosaccharide portion of a glycoconjugate, such as a glycoprotein, glycolipid, or a proteoglycan.

As used herein, an “antigen” is a substance that prompts the generation f antibodies and can cause an immune response. The terms “antigen” and “immunogen” are used interchangeably herein, although, in the strict sense, immunogens are substances that elicit a response from the immune system, whereas antigens are defined as substances that bind to specific antibodies. An antigen or fragment thereof can be a molecule (i.e., an epitope) that makes contact with a particular antibody. When a glycoprotein or a fragment thereof is used to immunize a host animal, numerous regions of the glycoprotein can induce the production of antibodies (i.e., elicit the immune response), which bind specifically to the antigen (given regions or three-dimensional structures on the glycoprotein).

As used herein, a “modification” is a substituent or a change in a substituent. A “substituent” is an atom r a group of atoms which replaces a hydrogen atom in a chemical structure.

The invention relates to an immunogenic glycan with a poly-β-1,4-ManNAc repeating-unit structure, modified with at least one 6-linked phosphoglycerol. Accordingly, in some embodiments, the invention may comprise a compound that comprises the glycan compound of Formula I, or an immunogenic part thereof, or an immunogenic analogue or modified form thereof:

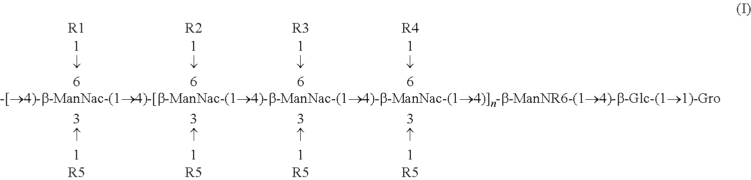

where n≥1, G1c represents glucose, ManNAc represents N-acctylmannosamine (2-acetamido-2,6-dideoxy-mannose), ManN represents mannosamine (2-amino-2-deoxy-D-mannopyranose), Gro represents glycerol, and where each of R1, R2, R3, R4 comprises any modification such as OH, phosphoethanolamine (PEtN) or phosphoglycerol (PGro), provided at least one of R1-R4 is —PGro; R5 comprises any modification such as —OH; and R6 comprises —H or —Ac. In one embodiment, one R5 in a terminal copy of the repeating structure may comprise a sugar, such as Ribf(ribofuranose).

In some embodiments, the glycan comprises a compound of Formula II, or an analogue or modified form thereof:

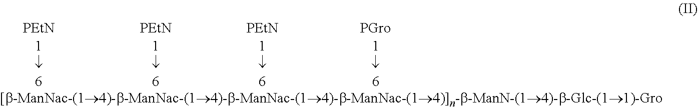

where n≥1.

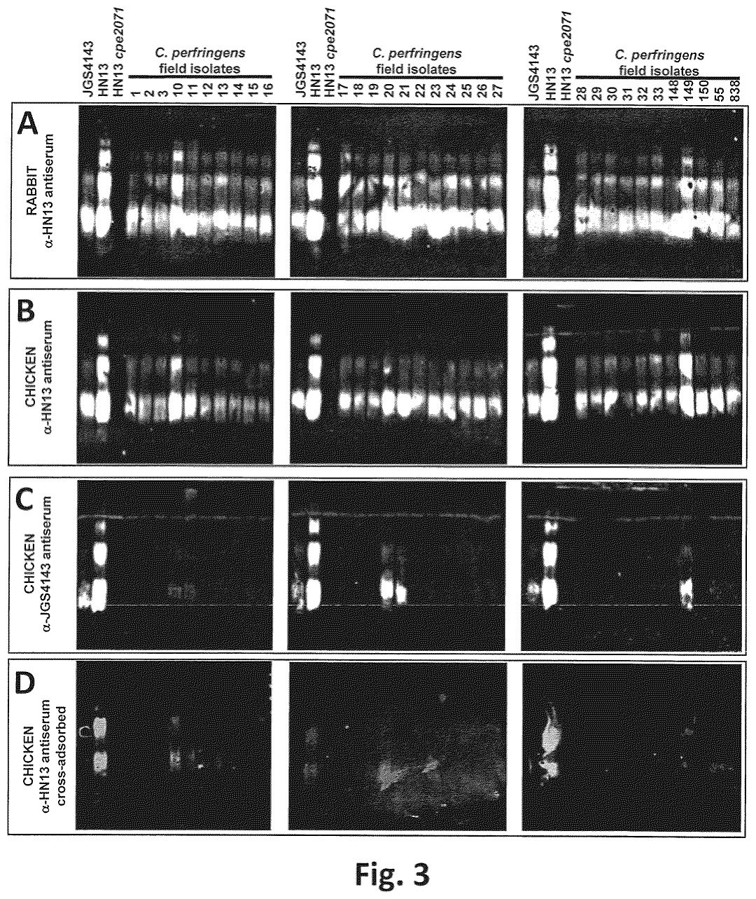

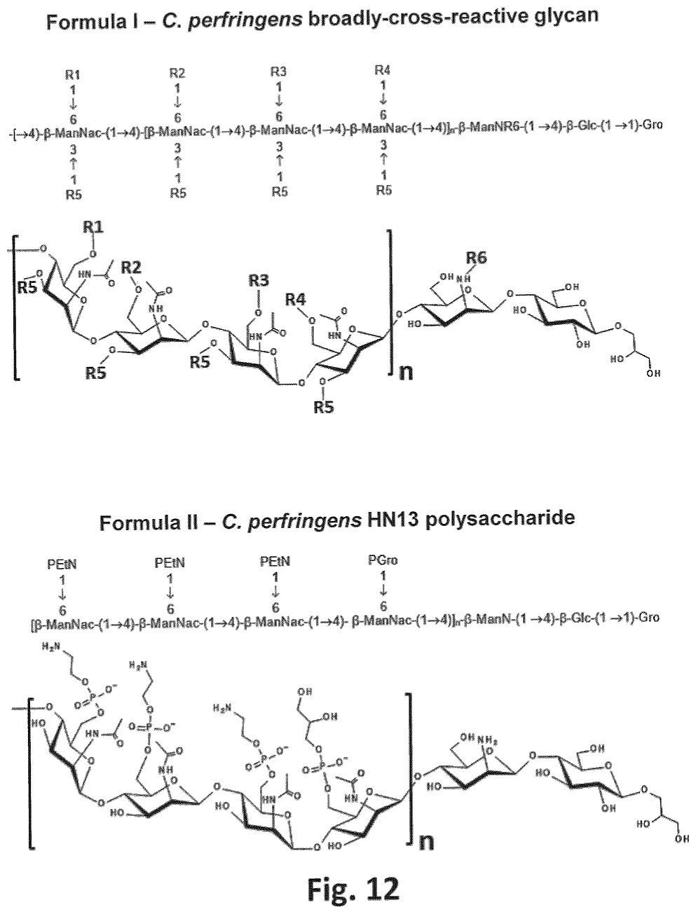

It is believed that one or more antigenic epitopes of the compound of Formula I are substantially conserved across C. perfringens isolates, as exemplified by cross-reactivity of antiserum raised against a surface polysaccharide of C. perfringens HN13 (Formula II—FIG. 12) that conforms to Formula I (Table 1; FIG. 3 panels A and B; FIG. 12), as compared to antigenic epitope(s) of the surface glycan from C. perfringens JGS4143 (Formula III—FIG. 12), which does not conform to Formula I. The glycan of Formula III is recognized by antiserum against HN 13 but elicits an immune response that is poorly cross-reactive with C. perfringens isolates (Table 1, FIG. 3 panel C; FIG. 12).

The immunogenic compound, analogue or modified form of Formula I or II is optionally connected or linked t a lipid, a single amino acid, an oligopeptide, a peptide, or a protein. The single amino acid may comprise asparagine, a serine or a threonine.

The conserved structure of Formulae I or II, or immunogenic analogues and modified forms thereof, contains all the features identified herein as necessary to elicit a cross-reactive immune response that recognizes a broad range of C. perfringens strains and is likely to be protective, based on the ability of antibodies against Formula I or II to protect chicks from C. perfringens-mediated mortality. As used herein, an “analogue” or “a modified form of a compound” is a compound which is substantially similar to another compound, where at least one component differs, but which is the functional equivalent of the other compound. In this case, the analogue or modified form will elicit an immune response which is cross-reactive with a compound of Formula I under suitable conditions, such as any of those described in the Examples below. As an example, the glycan of Formula III is not an analogue or modified form of Formula I or II, as elicits an immune response which is poorly cross-reactive with C. perfringens isolates. As an example, a compound which is an analogue or modified form of a glycan of Formula I or II will elicit an immune response which is reactive with at least 50%, or preferably at least 75%, and more preferably at least 90% of the field isolates identified in Table 1 below.

Any compound described r claimed herein may be chemically conjugated to a biornolecule, and/or expressed in an attenuated natural host or a heterologous host as an N-glycan, an O-glycan, on a lipid, on the bacterial surface, or on outer membrane vesicles (OMVs). Transfer to peptides can be mediated by an N—OTase or O—OTase co-expressed with the glycan, biosynthetic genes and an acceptor peptide, which transfer can occur in vivo or in vitro using purified components. If conjugated to a lipid, the lipid can be isolated and purified from a bacterial, archaeal or eukaryotic source or can be chemically synthesized. A linkage of the glycan compound to the lipid can be mediated through a phosphate, a pyrophosphate linker or by a glycosidic linkage.

For example, a carrier molecule may be linked to the immunogenic glycan by a covalent bond or an ionic interaction, either directly or using a linker. Linkage may be achieved by chemical cross-linking, e.g., a thiol linkage. A carrier protein or peptide may be linked to a glycan through, for example, O-linkage of the glycan to a threonine residue in the peptide. Methods for linking glycans to carrier molecules are well-known in the art, as are methods for preparing glycoconjugate vaccines. In some embodiments, a conjugated glycan antigen is prepared by conjugating a recombinantly-synthesized glycan to a carrier protein.

In another aspect, the invention may comprise a vaccine and a method for producing the vaccine, where the method comprises providing one or more of a glycan of Formula I or II and formulating into a vaccine composition. The glycan may be linked to a lipid, a single amino acid (such as asparagine, a serine or a threonine), an oligopeptide, a peptide, or a protein, and/or borne on an attenuated C. perfringens cell, or expressed on a bacteria engineered to heterologously express the glycan. Attenuated natural hosts may include inactivated cells or cells engineered to delete one or more toxins or other virulence factors (Thompson et al. 2006).

A vaccine is a preparation that can be administered to a subject to induce a humoral immune response (including eliciting a soluble antibody response) and/or cell-mediated immune response (including eliciting a cytotoxic T-lympocyte (“CTL”) response). The vaccines provided herein comprise an immunogenic glycan and are effective in inducing an immune response against the glycan antigen. The glycan may be in purified form, or conjugated to a biomolecule, or expressed and displayed by a host cell, as described above. As a result, the vaccines described herein are intended to induce an immune response against C. perfringens and provide protection from C. perfringens infections. Accordingly, the vaccine may be administered to any animal in need of protection from infection by C. perfringens, such as, without limitation, livestock such as cattle, sheep or poultry (turkeys, geese, ducks or chickens), canine or feline species, or humans.

Vaccines can further contain an adjuvant. The term “adjuvant” as used herein refers to any compound which, when injected together with an antigen, non-specifically enhances the immune response to that antigen. Exemplary adjuvants include Complete Freund's Adjuvant, Incomplete Freund's Adjuvant, Gerbu adjuvant (GMDP; C.C. Biotech Corp.), RIBI fowl adjuvant (MPL; RIBI Immunochemical Research, Inc.), potassium alum, aluminum phosphate, aluminum hydroxide, QS21 (Cambridge Biotech), Titer Max adjuvant (CytRx), Cystine phosphate Guanine (CpG) and Quil A adjuvant. Other compounds that can have adjuvant properties include binders such as carboxymethylcellulose, ethyl cellulose, microcrystalline cellulose, or gelatin; excipients such as starch, lactose or dextrins, disintegrating agents such as alginic acid, sodium alginate, Prinnogel, corn starch and the like; lubricants such as magnesium stearate or Sterotex; glidants such as colloidal silicon dioxide; sweetening agents such as sucrose or saccharin, a flavouring agent such as peppermint, methyl salicylate or orange flavouring, and a coloring agent.

Vaccines can be formulated using a pharmaceutically acceptable diluent. Exemplary “diluents” include water, physiological saline solution, human serum albumin, oils, polyethylene glycols, glycerine, propylene glycol or other synthetic solvents, antibacterial agents such as benzyl alcohol, antioxidants such as ascorbic acid or sodium bisulphite, chelating agents such as ethylene diamine-tetra-acetic acid, buffers such as acetates, citrates or phosphates and agents for adjusting the osmolarity, such as sodium chloride or dextrose. Exemplary “carriers” include liquid carriers (such as water, saline, culture medium, saline, aqueous dextrose, and glycols) and solid carriers (such as carbohydrates exemplified by starch, glucose, lactose, sucrose, and dextrans, anti-oxidants exemplified by ascorbic acid and glutathione, and hydrolyzed proteins.

Vaccines can contain an excipient. The term “excipient” refers herein to any inert substance (e.g., gum arabic, syrup, lanolin, starch, etc.) that forms a vehicle for delivery of an antigen. The term excipient includes substances that, in the presence of sufficient liquid, impart to a composition the adhesive quality needed for the preparation of pills or tablets.

Vaccines may be lyophilised or in aqueous form, e.g., solutions or suspensions. Liquid formulations of this type allow the compositions to be administered directly from their packaged form, without the need for reconstitution in an aqueous medium, and are thus ideal for injection. Compositions can be presented in vials, or they can be presented in ready filled syringes. The syringes can be supplied with or without needles. A syringe will include a single dose of the composition, whereas a vial can include a single dose or multiple doses (e.g. 2 doses).

Where a vaccine requires reconstitution, there is provided a kit, which can comprise two vials, or can comprise one ready-filled syringe and one vial, with the contents of the syringe being used to reconstitute the contents of the vial prior to injection.

The vaccine can be administered and formulated for administration by injection via the intramuscular, intraperitoneal, intradermal or subcutaneous routes; or via mucosal administration to the oral/alimentary, respiratory (e.g., intranasal administration), genitourinary tracts. Although the vaccine can be administered as a single dose, components thereof can also be co-administered together at the same time or at different times. In addition to a single route of administration, 2 different routes of administration can be used.

Another aspect of the application provides a method for immunizing an animal subject, comprising the step of administering an immunologically effective amount of the vaccine to a subject to produce an immune response. In one embodiment, the immune response comprises the production of bactericidal antibody production.

In other embodiments, there are provided compositions and methods for passive immunization comprising an antibody or an antigen-binding fragment thereof specific for any glycan described herein, which specifically binds to the glycan. As used herein, the term “antibody” refers to any immunoglobulin r intact molecule as well as to fragments thereof that bind to a specific antigen or epitope. Such antibodies include, but are not limited to polyclonal, monoclonal, chimeric, humanized, single chain, Fab, Fab′, F(ab′)2, F(ab)′ fragments, and/or F(v) portions of the whole antibody and variants thereof. All isotypes are emcompassed by this term, including IgA, IgD, IgE, IgG, and IgM. As used herein, the term “antibody fragment” refers to a functionally equivalent fragment or portion of antibody, i.e., to an incomplete or isolated portion of the fall sequence of an antibody which retains the antigen binding capacity (e.g., specificity, affinity, and/or selectivity) of the parent antibody. As is well known in the art, an antibody preparation may comprise monoclonal or polyclonal antibodies.

The terms “specific for” or “specifically binding” are used interchangeably to refer to the interaction between an antibody and its corresponding antigen. The interaction is dependent upon the presence of a particular structure of the compound recognized by the binding molecule (i.e., the antigen or epitope). In order for binding to be specific, it should involve antibody binding of the epitope(s) of interest and not background antigens, i.e., no more than a small amount of cross reactivity with other antigens (such as other proteins or glycan structures, host cell proteins, etc.). Antibodies, or antigen-binding fragments, variants or derivatives thereof of the present disclosure can also be described or specified in terms of their binding affinity to an antigen. The affinity of an antibody for an antigen can be determined experimentally using methods known in the art.

In another aspect, the invention may comprise diagnostic methods for detecting the presence of C. perfringens in a sample or a subject. In some embodiments, the methods of detecting the presence of C. perfringens in a subject comprise obtaining a biological sample from the subject and assaying the sample for the presence of the glycan described herein, wherein the presence of the glycan thereof in the sample indicates the presence of C. perfringens in the subject. In some embodiments, the assay comprises an immunoassay.

To gain a better understanding of the invention described herein, the following examples are set forth. It should be understood that these examples are for illustrative purposes only. Therefore, they should not limit the scope of this invention in any way.

### EXAMPLE 1

Clostridium strains were grown at 37° C. under anaerobic conditions in a Whitley DG250 Anaerobic Workstation (Don Whitley Scientific, Frederick, Md.) supplied with 5% hydrogen, 5% CO₂, 90% N₂) and propagated in PGY broth (3% proteose peptone #3, 2% dextrose, 1% yeast extract, 0.1% sodium thioglycollate) without agitation or on PGY agar (PGY broth containing 1.5% agar). Table 1 lists C. perfringens strains and isolates and derivatives thereof.

TABLE 1
C. perfringens strains.

| | | Reference/ |
|---|---|---|
| Strain | Description | Source |
| Clostridium cocleatum | Type strain | ATCC |
| ATCC | | |
| 29902 (NCTC 11210) | | |
| Clostridium perfringens | | ATCC |
| ATCC 43255 (VPI | | |
| 10463) | | |
| Clostridium perfringens | Chicken NE isolate | Barbara et al. (2008) |
| JGS4143 | | |
| Clostridium perfringens | highly transformable | Gohari et al. (2016) |
| SM101 | derivative of NCTC 8798 | |
| Clostridium perfringens | Type strain | |
| ATCC 13124 | | |
| Clostridium perfringens | | Nariya et al. (2011) |
| HN13 | | |
| cpe2071 (HLL8) | Transposon mutant; lacks | Liu et al. (2013) |
| | glycan of interest | |
| cpe2071 | Mutant complemented | This study |
| complemented | in trans | |
| cpe2237 | Chromosomal deletion | This study |
| | mutants; lack | |
| | putative PGro | |
| | transferase gene cpe2237 | |
| cpe2237 | Mutant complemented | This study |
| complemented | in trans | |
| Clostridium perfringens | | |
| field isolates | | |
| CP1 | chicken isolate | John Prescott |
| CP2 | chicken isolate | John Prescott |
| CP3 | chicken isolate | John Prescott |
| CP148 | Chicken NE, Quebec | John Prescott |
| CP149 | Chicken NE, Quebec | John Prescott |
| CP150 | Chicken NE, Quebec | John Prescott |
| isolate 6 (CP10) | chicken NE; ST02 | Chalmers et al. (2008) |
| isolate 9 (CP11) | chicken NE; ST04 | Chalmers et al. (2008) |
| isolate 10 (CP12) | chicken NE; ST03 | Chalmers et al. (2008) |
| isolate 14 (CP13) | chicken NE; ST05 | Chalmers et al. (2008) |
| isolate 15 (CP14) | chicken NE; ST06 | Chalmers et al. (2008) |
| isolate 19 (CP15) | chicken NE; ST08 | Chalmers et al. (2008) |
| isolate 20 (CP16) | chicken NE; ST09 | Chalmers et al. (2008) |
| isolate 23 (CP17) | chicken NE; ST10 | Chalmers et al. (2008) |
| isolate 28 (CP18) | chicken NE; ST13 | Chalmers et al. (2008) |
| isolate 30 (CP19) | chicken NE; ST14 | Chalmers et al. (2008) |
| isolate 32 (CP20) | chicken NE; ST15 | Chalmers et al. (2008) |
| isolate 42 (CP21) | chicken NE; ST16 | Chalmers et al. (2008) |
| isolate 57 (CP22) | chicken NE; ST22 | Chalmers et al. (2008) |
| isolate 18 (CP23) | chicken NE; ST08 | Chalmers et al. (2008) |
| isolate 22 (CP24) | chicken, healthy; ST01 | Chalmers et al. (2008) |
| isolate 26 (CP25) | chicken, healthy; ST11 | Chalmers et al. (2008) |
| isolate 27 (CP26) | chicken, healthy; ST12 | Chalmers et al. (2008) |
| isolate 34 (CP27) | chicken, healthy; ST10 | Chalmers et al. (2008) |
| isolate 60 (CP28) | chicken, healthy; ST06 | Chalmers et al. (2008) |
| isolate 16 (CP29) | chicken, healthy; ST07 | Chalmers et al. (2008) |
| isolate 45 (CP30) | chicken, healthy; ST17 | Chalmers et al. (2008) |
| isolate 46 (CP31) | chicken, healthy; ST19 | Chalmers et al. (2008) |
| isolate 47 (CP32) | chicken, healthy; ST18 | Chalmers et al. (2008) |
| isolate 54 (CP33) | chicken, healthy; ST20 | Chalmers et al. (2008) |
| JP55 | Equine NE | Gohari et al. (2016) |
| JP838 | Canine, haemorrhagic | Gohari et al. (2016) |
| | gastroenteritis | |
| Clostridium symbiosum | Type strain | ATCC |
| ATCC 14940 | | |

Whole cell lysates of C. perfringens, HN 13, JGS4143, and SM101 were generated for SDS-polyacrylamide gel electrophoresis (SDS-PAGE) and Western immunoblot analysis as follows: strains were streaked from −80° C. stocks onto PGY agar plates (with antibiotics as appropriate), and grown overnight. For each strain, a single colony was used to inoculate 10 ml of PGY broth, allowed to grow for 6 h, harvested by centrifugation (13000 ×g, 10 min), washed with phosphate buffered saline (PBS) and resuspended in PBS to OD₆₀₀ₙₘ=2.0. Cells from 1 ml were harvested by centrifugation as above, resuspended in 100 μl of PBS, and incubated with 2 mg ml⁻¹ lysozyme at 37° C. for 1 h. Each sample was combined with 67 μl of 4× SDS-PAGE sample buffer (Laemmli (1970)), heated to 95° C. for 10 min, allowed to cool, then either analyzed by SDS-PAGE according to the method of Laemmli (Laemmli (1970)) or incubated with 0.5 mg ml⁻¹ proteinase K at 55° C. for 1 h prior SDS-PAGE analysis. Following electrophoresis, samples were transferred electrophoretically to 0.2 μm nitrocellulose membrane (Bio-Rad Laboratories Canada, Mississauga, ON) and subjected to Western immunoblot analysis (Burnette (1981)) using polyclonal rabbit antiserum raised against whole cells of C. perfringens HN13 (Dr. S.G. Melville, Virginia Tech) as the primary (1:1000 dilution), and IRDye 680RD goat anti-rabbit IgG (LI-CUR Biosciences, Lincoln, Nebr.) as the secondary antibody (1:15,000), and visualized on a LI-CUR Odyssey infrared imaging system (LI-COR Biosciences).

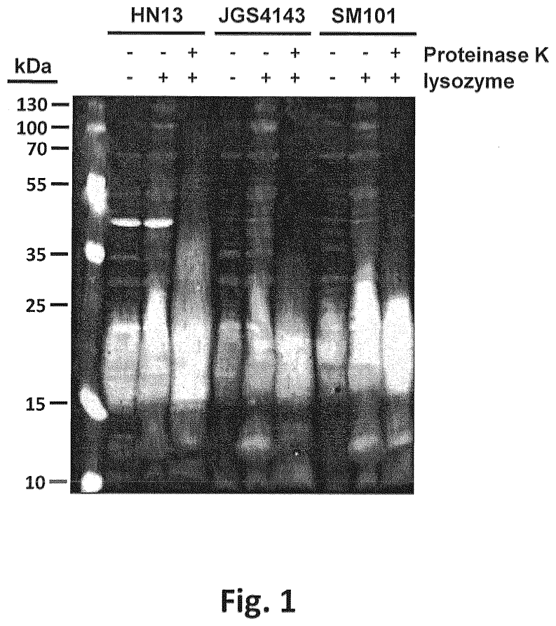

FIG. 1 shows a Western immunoblot of whole cell lysates of the C. perfringens HN13, J054143, and SM101 strains using rabbit antiserum that was raised against whole cells of C. perfringens HN 13.

The reactivity in all strains was similar, with a large antigen “smear” and a few high molecular weight bands present irrespective of lysozyme treatment. Treatment of proteinase K resulted in loss of the few high molecular weight bands but the large “smear” reactivity was unaffected, indicating that the antigen responsible is not protein-based, suggestive that the antigen is a polysaccharide, glycolipid, or lipid molecule.

Thus, it appears that C. perfringens likely produces a non-protein antigenic molecule that dominates the immune response.

### EXAMPLE 2

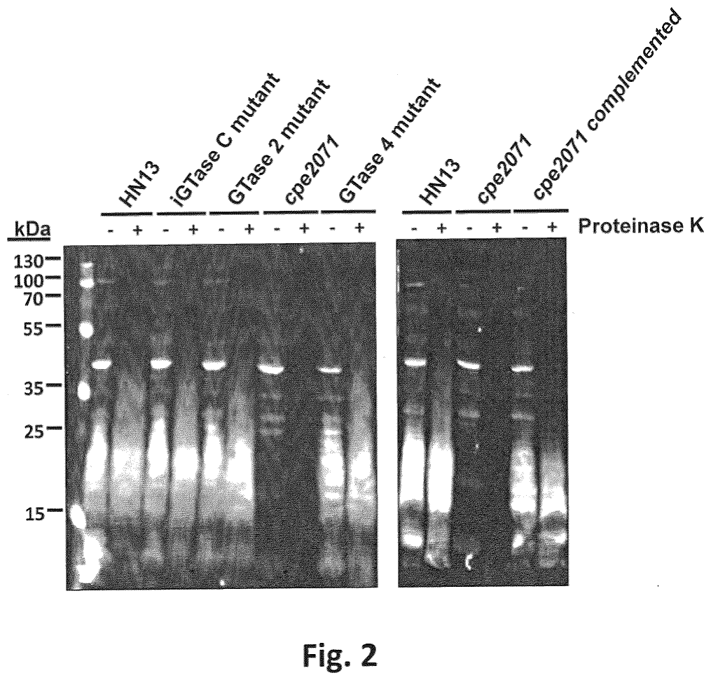

FIG. 2 depicts an anti-C. perfringens Western immunoblot of whole cell lysates with and without proteinase K treatment from HN13, four different glycosyltransferase transposon mutants, and the cpe2071 glycosyltransferase mutant complemented with the plasmid-borne cpe2071 gene (prepared as described in Example 1). Whole cell lysates of four glycosyltransferase mutants (isolated from a previously described C. perfringens HN13 transposon library (Liu et al. (2013)) were analyzed by Western immunoblotting and lysates from a mutant with the cpe2071 gene disrupted (strain HLL8) did not contain the proteinase K-resistant antigen observed in the wild-type strain. Complementation of this mutant with a plasmid-borne copy of the cpe2071 gene resulted in restoration of the proteinase K-resistant antigen confirming that loss of this antigen in the cpe2071 mutant was due to disruption of the cpe2071 gene. Given that cpe2071 encodes a polysaccharide and the antigen is proteinase K-resistant, the antigen is either a polysaccharide or a polysaccharide-containing glycolipid.

Thus, according to this example, it appears that the immunodominant surface antigen of C. perfringens is likely a polysaccharide or glycolipid with a polysaccharide component.

### EXAMPLE 3

Formalin-fixed C perfringens HN13 and JGS4143 cells were prepared as follows for intramuscular (IM) injection into chickens. Cells were grown overnight on PGY agar plates as described in Example 1. Cells from one plate each were harvested and resuspended in 10 ml PBS, pelleted by centrifugation, resuspended in 10 ml PBS containing 1% (v/v) formalin, and incubated at 4° C. for 2 h. Cells were washed 4 times in 2 ml of PBS to remove formalin, and resuspended in PBS to an OD₆₀₀ₙₘ of 1.0. The cell suspension was mixed 1:1 with either Freund's Complete adjuvant (FCA, primary injection) or Freund's Incomplete adjuvant (FIA, boost injection). Primary injections (150 μl×2, IM in the breast muscle) were given to broilers at 7 days of age, followed by boost injections (150 μl×2, IM in the breast muscle) at 21 days of age. Chickens were culled on Day 35 and exsanguinated. Blood was allowed to clot at room temperature overnight, and the next day the samples were centrifuged at 13 000 ×g and the serum was aspirated by pipette and stored at 4° C.

A total of 32 field isolates of C. perfringens were obtained from Dr. John Prescott (University of Guelph, Guelph, ON, Canada), consisting of isolates from both healthy and NE chickens covering a range of Multi-Locus Sequence Typing sequence types (ST), as well as two strains isolated from non-chicken infections (equine NE and canine haemorrhagic gastroenteritis) (Table 1).

Whole cell lysates of C. perfringens for SDS-polyacrylamide gel electrophoresis (SDS-PAGE) and Western immunoblot analysis were prepared by boiling cells in SDS-PAGE buffer, treating with proteinase K, and boiling in SDS-PAGE buffer (as described in Example 1), then separated by SDS-PAGE and analyzed by Western immunoblotting using rabbit anti-C. perfringens antiserum (which was raised against C. perfringens HN13) as well as the chicken anti C, perfringens antisera raised against C. perfringens HN13 and JGS4143 (described above). To remove undesirable signals from antigens other than the glycan of interest, the rabbit and chicken antisera raised against C. perfringens HN13 were adsorbed against whole cells of the C. perfringens HN13 cpe2071 mutant (strain HLL8), which does not make the glycan of interest. The chicken antiserum raised against C. perfringens JGS4143 was used without any adsorption step since no glycan-minus mutant was available in that background. The adsorption was performed in the following manner: C. perfringens HN13 cpe2071 was grown as described for whole cell lysates, washed with PBS and adjusted to OD₆₀₀ₙₘ =1.0 in PBS, 4×1-ml aliquots were pelleted by centrifugation as described above. The first aliquot was resuspended in 100 μl. of either rabbit or chicken anti-C. perfringens HN13 antiserum, allowed to incubate at room temperature for 1 h, pelleted by centrifugation, and the supernatant was decanted. This process was repeated sequentially for each of the 3 remaining cell aliquots using the supernatant from the previous round to resuspend the cells. This adsorbed antiserum was used as the primary antibody and 1RDye 680RD goat anti-rabbit IgG was used as the secondary antibody as was described in Example 1.

FIG. 3 depicts Western immunoblots of whole cell lysates from C. perfringens field isolates vs JGS4143 and HN13 (+ve controls) and the HN13 cpe2071 mutant (−ve control) using the adsorbed rabbit and chicken anti-C. perfringens HN13 antisera as well as the unadsorbed anti-C. perfringens JGS4143 antisera. For both the rabbit and chicken antisera raised against C. perfringens HN13, all of the strains showed reactivity similar to HN13 and JGS4143, indicating that these strains produce a similar or closely related glycan compared to C. perfringens HN13. Note that reactivity consistent with the glycan of interest was observed in field isolates from both NE and healthy chickens, as well as from an equine NE (JP55) and a canine haemorrhagic gastroenteritis (JP838) isolate, indicating that the glycan of interest is present on isolates of C. perfringens irrespective of the host species or the disease state of the host animals. In contrast, the chicken antiserum raised against C. perfringens JGS4143 was reactive with both the HN13 and JGS4143 lysate controls, but only 5 of the field isolates showed reactivity, with 3 isolates (20, 21, and 149) showing moderate reactivity and a further 2 field isolates (10 and 11) only faintly reactive.

Thus, it appears that the surface polysaccharide antigen from C. perfringens HN13 is a specific example of a glycan conforming to Formula 1 herein (FIG. 12), and is either broadly conserved or has one or more epitopes that elicit a broadly cross-reactive immune response, while the surface polysaccharide antigen from C. perfringens JGS4143 (FIG. 12) is far less cross-reactive in exemplary field isolates of C. perfringens.

### EXAMPLE 4

Proteinase K-treated cell lysates of Clostridium cocleatum, Clostridium perfringens, and Clostridium symbiosum were prepared in the same manner as described for C. perfringens cell lysates in Example 1. The non-C. perfringens lysates, along with JGS4143 and HN lysates as positive controls and the HN13 cpe2071 mutant lysate as a negative control, were separated by SDS-PAGE and analyzed by Western immunoblotting using rabbit anti-C. perfringens antiserum adsorbed against whole cells of the C. perfringens HN13 epe2071 mutant as described in Example 3.

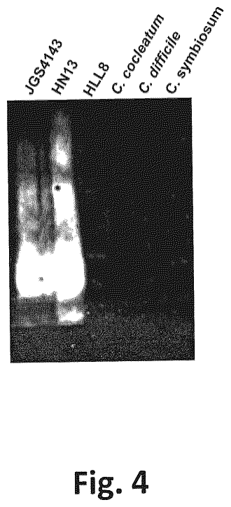

FIG. 4 depicts Western immunoblots of whole cell lysates from representative strains of C. coeleatum, C. perfringens, and C. symbiosum vs JGS4143 and HN13 (+ve controls) and the HN13 cpe2071 mutant eve control) using anti-C. perfringens rabbit antiserum adsorbed against whole cells of the HN13 cpe2071 mutant. None of the non-C. perfringens lysates displayed reactivity consistent with the glycan of interest, indicating that the conserved C. perfringens antigen is not present in these related Clostridium strains.

Thus, according to this example, it appears that the conserved C. perfringens antigen is likely not present in other Clostridium species.

### EXAMPLE 5

For passive protection experiments, leghorn chicks were challenged at 1 day of age with C. perfringens in the presence and absence of chicken anti-C. perfringens antiserum as follows. To prepare the oral gavage solutions, the chicken NE strain C. perfringens JGS4143 was streaked on PGY agar the day before gavage (day 0) and grown overnight as described above. On the day of gavage (day 1), the cells were harvested in PBS, pelleted by centrifugation at 13,000 x g for 30 min, and washed twice with PBS. The washed cell pellet was resuspended to ˜3.7×10⁹ cells per ml in PBS, and separately a 1/10 dilution of the highly cross-reactive chicken anti-C. perfringens HN13 antiserum in PBS was prepared. The C. perfringens JGS4143 cell suspension was then mixed 9:1 with either PBS or the diluted chicken anti-C. perfringens antiserum immediately prior to gavage, as appropriate. In total, 9 birds were orally gavaged with 300 μl of the C. perfringens/PBS mixture without antiserum (1×10⁹ cells), 9 birds were orally gavaged with 300 μl of the C. perfringens/PBS mixture containing antiserum (1 x 10⁹ cells), and 5 birds were orally gavaged with PBS alone as a control, and bird mortality was monitored over 7 days.

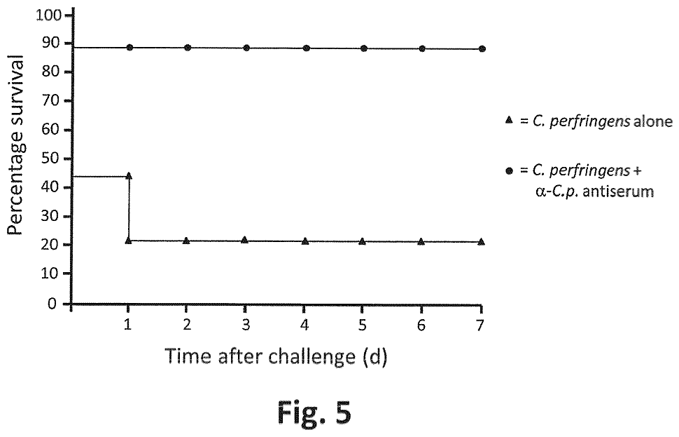

FIG. 5 depicts the percent survival of birds in the groups orally gavaged with C. perfringens JGS4143 alone, and co-gavage with JGS4143 with a 1:100 dilution f anti-C. perfringens antiserum. Seven days post-gavage, 100% of birds orally gavaged with PBS alone survived (not shown), only 22% survival (2 of 9 birds) was observed in the group gavaged with C. perfringens alone, and an 89% survival rate (8 of 9 birds) was observed in the group co-gavaged with C. perfringens and 1:100 anti-C. perfringens antiserum.

### EXAMPLE 6

For opsonophagocytosis assays, C. perfringens JGS4143 cells were incubated with heparinized chicken blood and either nave chicken serum or anti-C. perfringens HN13 antiserum according to the method previously described by Guyette-Desjardins et al (2016) with modifications, as follows. To prepare the bacterial cells for this assay, the chicken NE strain C. perfringens JGS4143 was streaked on PGY agar the day before the cull of a 5-week old broiler chicken (day 34) as a source of fresh chicken blood, and grown overnight as described above. On the day of cull and blood collection (day 35), the cells were harvested in PBS, pelleted by centrifugation at 13,000×g for 30 min, and washed twice with PBS. The washed cell pellet was resuspended to ˜2.9×10⁵ cells per ml in RPMI 1640 media supplemented with 5% heat inactivated chicken serum, 10 mM HEPES, 2 mM L-glutamine, and 50 μM β-mercaptoethanol, and blood from a single culled chicken was collected in a heparin-coated tube to prevent coagulation. The heparinized blood was diluted ⅓ in the supplemented RPM! 1640 listed above. The diluted blood (50 μl) was combined with 40 μl of either naïve chicken serum or chicken anti-C. perfringens HN13 antiserum in a microtube, followed by addition of 10 of the C. perfringens JGS4143 suspension, resulting in an approximate MOI of 0.015 based on 2.9×10³ bacterial cells in the reaction and a calculated leukocyte content of 1.9×10⁵ leukocytes based on literature values of leukocytes in the blood of broiler chickens (Orawan and Aengwanich (2007)). The tops of the tubes were pierced using a sterile 25-gauge needle and then placed in a 5% CO₂ incubator at 37° C. for 2 h, after which each reaction was combined with 80% sterile glycerol and incubated at −80° C. until ready to be plated. To enumerate the cells in each reaction, samples were thawed on ice, and 100 μl aliquots of 10-fold serial dilutions were plated n PGY agar and incubated under anaerobic conditions for 18 h. Percent bacterial killing values were calculated using the following formula: % bacteria killed=[(# of cells in naïve chicken serum reaction−# of cells recovered in the reaction of interest)/(# of cells in naïve chicken serum reaction)]×100,

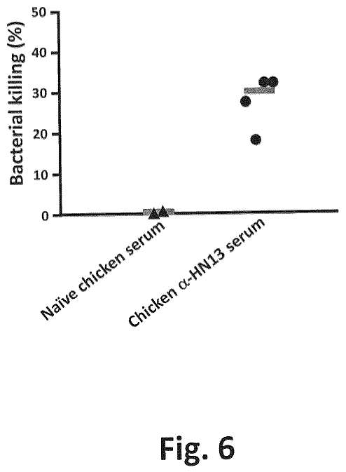

FIG. 6 depicts the percent bacterial killing observed in opsonophagocytosis assay reactions containing chicken anti-C. perfringens HN13 antiserum, with an observed median % bacterial killing of C. perfringens JGS4143 of 29.5% with this serum.

### EXAMPLE 7

For NMR experiments, Clostridium strains were grown in PGY broth at 37° C. with agitation at 50 rpm in a BioFlo 115 Fermenter (Eppendorf, Mississauga. ON) that was supplied with N₂ at a flow rate of 1L/rnin. The media were pre-warmed and conditioned with N₂ for 1 h prior to inoculation with a 40-ml overnight broth culture. Where appropriate, media were supplemented with 30 μg ml⁻¹ erythromycin (Em).

The polysaccharide from C. perfringens was extracted and purified from 10-L fermenter cultures of C. perfringens HN13 and JGS4143 as follows: cultures were inoculated with a 40 ml O/N culture and allowed to grow 6 h (˜OD 2.0) before harvesting by centrifugation (13,000 ×g. 30 min). Cells were washed once with PBS, resuspended in 400 ml of MilliQ water, and boiled for 30 min with stirring on a hot plate. The mixture was cooled, cells were pelleted by centrifugation (as above), the supernatant was removed, and the pellet was subjected to phenol:hot water extraction according to the method of Westphal and Jann (1965) with modifications. The pellet was resuspended in 200 ml of saline (125 mM NaCl) and combined with 200 ml of liquified phenol preheated in a 70° C. water bath, and the mixture was incubated with stirring for 1 h. The mixture was cooled on ice, centrifuged (13,000 ×g for 30 min) to separate the aqueous and phenol phases, and the phenol phase was dialyzed against tap water for 5 days and then lyophilized. The lyophilized sample was resuspended in 100 ml MilliQ water, subjected to centrifugation at 13,000 ×g for 30 min, and then placed in an ultracentrifuge for 16 h. After removing the supernatant, the clear pellet was resuspended again in MilliQ water and re-pelleted by ultracentrifugation (as above) to remove residual traces of the supernatant, resuspended in 20 ml of MilliQ and lyophilized. The isolated compounds used for NMR. were compared to the proteinase K-resistant antigenic molecules as observed in Western immunoblots.

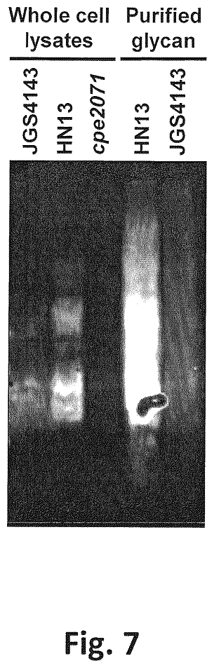

FIG. 7 depicts a Western immunoblot of the purified antigens in comparison to proteinase K digested whole cell lysates of HN13 and MS4143 (+ve controls) and the HN13 cpe2071 mutant (-ve control) using rabbit antiserum raised against C. perfringens HN13.

### Glycosyl Composition Analysis of ohe Purified Surface Polysaccharides from C. perfringens HN13 And JGS4143

The composition of the glycolipids isolated from these two stains (as described above) was determined by combined gas chromatography/mass spectrometry (GC-MS) of per-O-trimethylsilyl derivatives of the monosaccharide methyl glycosides produced by acid methanolysis of the samples as described by Santander et al. (2013). Briefly, lyophilized HN13 and JGS4143 glycolipids were heated with methanolic HCl in a sealed screw-top glass test tube for 18 h at 80° C. After cooling and removal of the solvent under a stream of nitrogen, the samples were treated with a mixture of methanol, pyridine, and acetic anhydride for 30 min. The solvents were evaporated, and the samples were derivatized with Tri-Sil® (Pierce) at 80° C. for 30 min. GC/MS analysis of the TMS methyl glycosides was performed on an Agilent 7890A GC interfaced to a 5975C MSD, using an Supelco Equity-1 fused silica capillary column (30 m×0.25 mm ID).

Glycosyl composition analysis showed that HN13 polysaccharide contains glycerol (Gro), glucose (G1c), traces of N-acetylmannosarnine (ManNAc) and fatty acids: C20, C18, C16 and C14. The JG4143 polysaccharide contains ribose (Rib), glucose (G1c), traces of N-acetylmannosamine (ManNAc) and fatty acids: C20, C18 and C16. As shown and described below, the major glycosyl residue in the glycolipid is ManNAc, however, it is largely not observed using this method due to the majority of these residues being substituted with phosphoethanolamine or phosphoglyccrol (see below).

To prepare samples for NMR spectroscopy, all purified glycolipids were deacylated as follows: lyophilized samples were dissolved in in concentrated NH₄OH, incubated at 80° C. for 1 h, allowed to cool, and lyophilized. The lyophilized material was dissolved in distilled water and fractionated on a BioGel P6 column using deionized water as the eluent. Fractions were collected based on response from a refractive index detector, lyophilized, and then washed 3 times with dichloromethane to completely remove free fatty acids from the samples.

For all NMR experiments, lyophilized samples were dissolved in 0.2 ml D₂O, and transferred to a 3 mm OD NMR tube. 1D proton spectra were acquired at 25° C. with standard “Presat” solvent signal suppression on a Varian 600 MHz spectrometer equipped with 3 ram cold probe (Varian, Inova Palo Alto, Calif.). All spectra were acquired with standard Varian pulse sequences. The NMR acquisitions were processed using MNova software (Mestrelab Research, Spain). The spectra were referenced relative to the DSS signal (δ_{H}=0 ppm; δ_{C}=0 ppm).

### NMR spectroscopy of the surface carbohydrate from C. perfringens HN 13

Delipidated HN13 polysaccharide was analyzed by 1D/2D NMR spectroscopy; proton, HSQC, COSY, TOCSY, and NOESY analyses. This allowed assignment of the proton and carbon chemical shifts of each residue, and also the determination of their linkages, sequence and the substitution positions of the PEtN and PGro substituents. The chemical shift assignments are given in Table 2 below.

TABLE 2
¹H and ¹³C NMR chemical shifts of the HN13 polysaccharide, recorded in D₂O at 30° C.

| Residue | H-1/C-1 | H-2/C-2 | H-3/C-3 | H-4/C-4 | H-5/C-5 | H-6/C-6 |
|---|---|---|---|---|---|---|
| -4)-β-ManNAc6PEtN-(1- | 4.87 | 4.61 | 3.94 | 3.80 | 3.61 | 4.12 |
| A | 101.1 | 54.0 | 71.7 | 77.9 | 74.9 | 65.4 |
| -4)-β-manNAc6PGro-(1- | 4.89 | 4.58 | 3.94 | 3.80 | 3.61 | 4.19 |
| B | 101.1 | 54.1 | 71.7 | 77.9 | 74.9 | 65.4 |
| PEtN | 4.11 | 3.23 | — | — | — | — |
| | 63.3 | 41.4 | | | | |
| PGro | 3.86; 3.91 | 3.94 | 3.60; 3.66 | — | — | — |
| | 67.6 | 71.7 | 63.4 | | | |

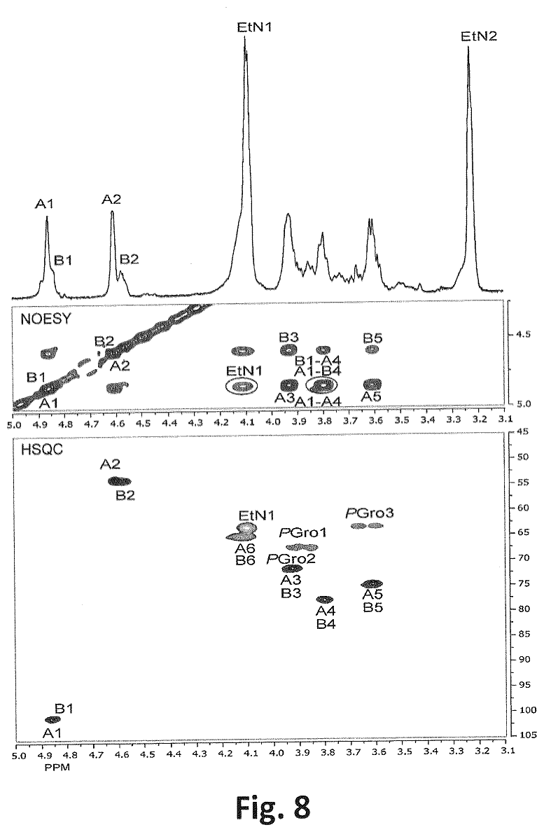

FIG. 8 depicts the 1_{H} NMR, NOESY (200 ms) and gHSQC spectra (D₂O, 30° C.) of the deacylated polysaccharide from Clostridium perfringens HN 13.

The 1_{H} NMR spectrum (FIG. 8, top) contained two anomeric signals at δ4.87 (residue A) and δ4.84 (residue B) in the ratio 3:1, which were both due to β-ManpNAc residues as indicated by their respective downfield H-2 chemical shifts, δ4.61 and δ4.58, and C-2 chemical shifts at δ54.0 and 54.1 (FIG. 3, middle and bottom). The high-field signal at δ2.06, was assigned to the NAc groups attached to the C-2 of each ManNAc residue. The strong intraresidual NOE correlations (FIG. 3, middle) between H-1 and H-3, and between H-1 and H-5 confirm the 13-configuration of these residues. All ManNAc residues were connected by (1→4) linkages, and were substituted at O-6 by PEtN (residue A) or PGro (residue B). The 4-and 6-substitution f the residues A and B, respectively, were supported by their 13_{C} chemical shifts (FIG. 8, bottom; Table 2): A C-4 δ77.9, A C-6 δ65.4, B C-4 δ77.9, B C-6 δ65.4. The fact that a terminal residue was not observed indicates that the polysaccharide has a high molecular weight.

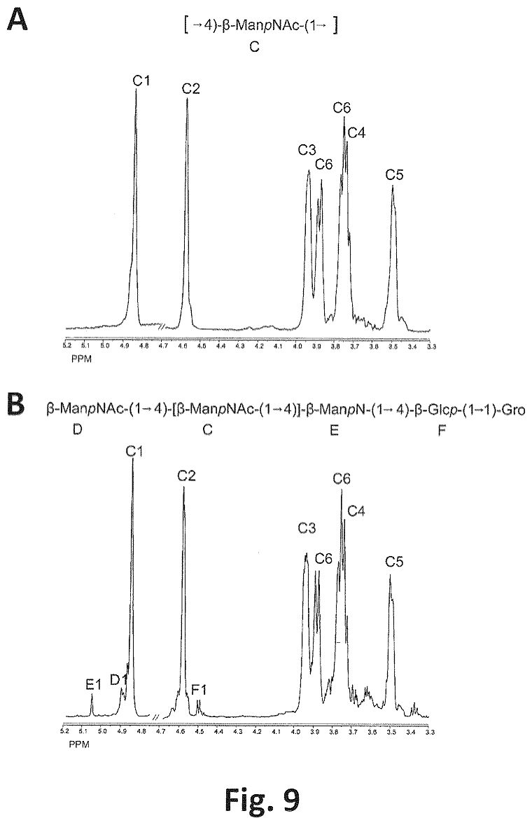

In order to get more information about the structure, the HN 13 polysaccharide was dephosphorylated by dissolving the lyophilized delipidated sample in 48% HF and incubating at 4° C. for 48 h, followed by evaporation of the sample on ice and lyophilized once more. The generated product mixture was subjected to size exclusion chromatography by Bio-Gel P6 column and two fractions, denoted F1 and F2, were obtained. The 1D/2D NMR analysis allowed proton and carbon assignments of the residues in both F1and F2 as well as the linkage and sequence of these residues (FIG. 9; Table 3)

TABLE 3
¹H and ¹³C NMR chemical shifts of the dephosphorylated HN13
polysaccharide (Fraction F1-F2), recorded D₂O at 25° C.

| Residue | H-1/C-1 | H-2/C-2 | H-3/C-3 | H-4/C-4 | H-5/C-5 | H-6/C-6 |
|---|---|---|---|---|---|---|
| -4)-β-ManNAc-(1- | 4.84 | 4.57 | 3.93 | 3.73 | 3.49 | 3.76; 3.88 |
| C* | 100.6 | 54.0 | 71.6 | 77.3 | 76.3 | 61.1 |
| T-β-ManNAc-(1- | 4.85 | 4.55 | 3.82 | 3.51 | 3.43 | 3.80; 3.92 |
| D | 100.6 | 54.4 | 73.0 | 67.8 | 77.7 | 61.5 |
| -4)-ManN-(1- | 5.03 | 3.91 | 4.11 | 3.79 | 3.57 | 3.76; 3.89 |
| E | 97.6 | 55.2 | 69.4 | 76.6 | 75.9 | 61.1 |
| -4)-β-Glc-(1- | 4.48 | 3.36 | 3.68 | 3.76 | 3.59 | 3.73; 3.89 |
| F | 103.5 | 74.1 | 74.9 | 79.1 | 75.6 | 61.1 |
| Gro | 3.76; 3.92 | 3.93 | 3.60; 3.67 | | | |
| | 72.0 | 71.9 | 63.5 | | | |

*Fraction, F1 , contained only these chemical shifts.

FIG. 9 depicts the 1_{H} NMR spectra (D₂O, 25° C.) of the F1 and F2 fractions from Bio-Gel P6 chromatography of dephosphorylated HN13 polysaccharide. These data show that the backbone of the dephosphorylated polysaccharide, F1, contains only linear chains of (β-4-linkedManNAc (C) residues. All PEtN or PGro groups had been removed by the HF treatment. The 1D/2D NMR analysis of the low molecular fraction (F2), showed that HN13 polysaccharide has a →4-β-ManN-(1→4)-β-G1c-(1→1)-Gro component at its reducing end followed by β-4-linked ManNAc residues. MALDI-TOF-MS analysis, together with the above NMR data, confirmed that fraction F2 contained the above trisaccharide component followed by successive elongation with 13-4-linked ManNAc residues (Table 4).

TABLE 4
Observed masses and proposed compositions for ions
generated by positive-ion mode on Bio-Gel P6 fraction F2.

| Proposed structure | Observed (m/z) |
|---|---|
| ManNAc₃ManNGlcGro | 1047.5 [M + Na]⁻ |
| ManNAc₄ManNGlcGro | 1250.6 [M − Na]⁻ |
| ManNAc₅ManNGlcGro | 1453.7 [M − Na]⁻ |
| ManNAc₆ManNGlcGro | 1656.8 [M + Na]⁻ |
| ManNAc₇ManNGlcGro | 1859.8 [M + Na]⁻ |

Combined, these data indicate that the HN13 polysaccharide is comprised of a repeating polymer of ManNac residues modified with PGro or PEtN in a 1:3 ratio linked to ManN-G1Gro at the reducing end (FIG. 12), with a structure of Formula II (shown above).

### NMR spectroscopy of the Surface Carbohydrate from C. perfringens JGS4143

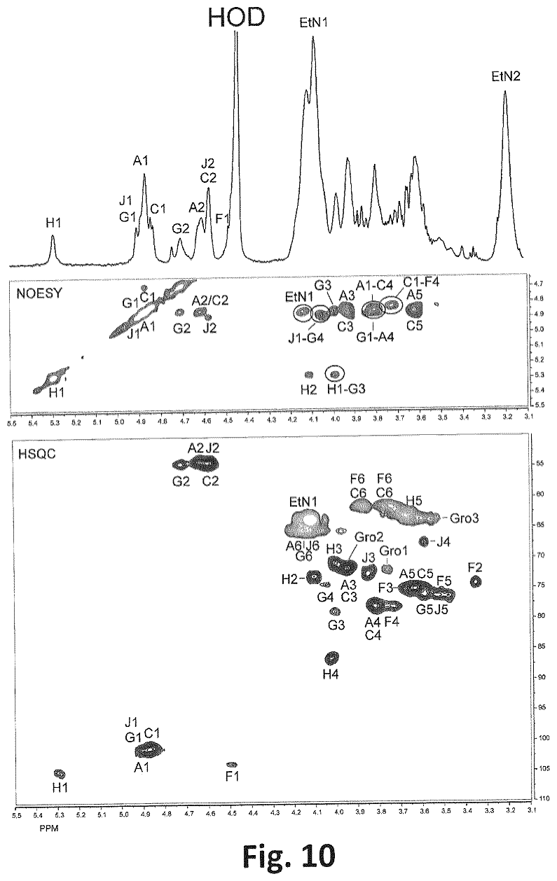

1D and 2D NMR analysis (as described for the HN13 polysaccharide) allowed complete assignment of the protons and carbons for these residues (FIG. 10; Table 5).

FIG. 10 depicts the 1_{H} NMR, NOESY (200 ins) and gHSQC spectra (D₂O, 60° C.). The 1_{H} NMR spectrum of JGS4143 polysaccharide showed the presence of spin systems belonging to: →4)-β-ManpNAcPEtN-(1→(residue A); →4)-β-ManpNAc-(1→(residue C); →4)-β-G1cp -(1→(residue F); →3,4)-β-ManpNAcPEtN-(1 (residue G); T-α-Ribf-(1→(residue H); T-β-ManpNAcPEtN-(1→(residue J).

TABLE 5
¹H and ¹³C NMR chemical shifts of the JG54143 polysaccharide, recorded in D₂O at 30° C.

| Residue | H-1/C1 | H-2/C-2 | H-3/C-3 | H-4/C-4 | H-5/C-5 | H-6/C-6 |
|---|---|---|---|---|---|---|
| -4)-β-ManNAc6PEtN-(1- | 4.87 | 4.61 | 3.94 | 3.82 | 3.62 | 4.12 |
| A | 101.1 | 54.0 | 71.7 | 77.9 | 74.9 | 65.2 |
| -4)-βp-ManNAc-(1- | 4.84 | 4.57 | 3.93 | 3.73 | 3.55 | 3.75; 3.87 |
| C | 100.7 | 54.0 | 71.6 | 77.9 | 76.1 | 61.2 |
| -4)-β-G16-(1- | 4.49 | 3.35 | 3.68 | 3.73 | 3.59 | 3.75; 3.87 |
| F | 103.4 | 73.9 | 74.8 | 77.8 | 76.1 | 61.2 |
| -3,4)-β-ManNAc6PEtN-(1- | 4.87 | 4.72 | 4.00 | 4.04 | 3.63 | 4.12 |
| G | 100.8 | 54.0 | 78.6 | 74.1 | 75.0 | 65.2 |
| T-β-Ribf-(1- | 5.29 | 4.10 | 4.00 | 4.02 | 3.66; 3.71 | |
| H | 104.6 | 72.8 | 70.5 | 86.2 | 62.6 | |
| T-β-ManNAc6PEtN-(1- | 4.90 | 4.59 | 3.85 | 3.59 | 3.58 | 4.12 |
| J | 101.1 | 54.3 | 72.8 | 67.4 | 76.2 | 65.2 |
| Gro | 3.75; 3.93 | 3.94 | 3.55; 3.64 | | | |
| | 71.7 | 71.7 | 63.5 | | | |
| PEtN | 4.11 | 3.20 | | | | |
| | 63.4 | 41.4 | | | | |

These data, unlike those for the HN13 polysaccharide, allowed the identification of a terminal ManNAc residue as well as the reducing end -4)-β-G1cp-(1→1)-Gro component. Comparison of these NMR data with those for the HN13 polysaccharide (described above) showed that molecule was an oligosaccharide with a β-4-linked ManNAc backbone that was largely substituted by PEtN, as was the case for the HN13 polysaccharide, but significantly differed from the HN13 polysaccharide in that it was devoid of PGro and contained α-Ribf substituted at O-3 of one of the ManNAc residues.

These data indicate that the JGS4143 polysaccharide is comprised of a repeating polymer of ManNac residues modified with PEtN and linked to ManN-G1c-Gro at the reducing end, similar to the polysaccharide of HN13, but devoid of the Pao modifications observed in the HN13 polysaccharide and having an additional branching a-Ribf residue at O-3 on the ManNAc residue proximal to the terminal ManNAcPEtN residue (FIG. 12), with a structure of Formula

Combined, these data indicate that C. perfringens strains produce a common class of surface polysaccharides, and that the surface polysaccharide from C. perfringens HN13 is a glycolipid with a long polysaccharide chain with a repeating-unit structure of 1,4-linked ManNAc modified with PGro or PEtN in a 1:3 ratio that contains one or more epitopes shared with all C. perfringens strains tested to date. In contrast, C. perfringens JGS4143 produces a related glycolipid that fractionates similarly and whose polysaccharide backbone is also a polymer of 1,4-linked ManNAc residues modified with PEtN, but differs from the HN13 glycan primarily by the absence of PGro modifications and shorter polymer length.

The ability of the HN13 glycan to elicit an immune response (in both rabbits and chickens) that is broadly cross-reactive to all C. perfringens field isolates tested, contrasted with the non-cross-reactive JOS glycan (eliciting an immune response in chickens that is only cross-reactive with 16% of field isolates tested), taken with the structural features of the solved structures for both glycans, suggests that the broadly cross-reactive immune response to the HN13 is dependent on at least the presence of at least one PGro modification, and possibly the absence of the pentose (α-Ribf) observed in JGS4143.

### EXAMPLE 8

For generation of a C. perfringens HN13 mutant that lacks the phosphoglycerol moiety, putative phosphoglycerol transferase genes were identified by surveying the genome of C. perfringens strain 13 (taxid:195102) for genes annotated to potentially have a role in LTA biosynthesis or transfer of phosphoglycerol, followed by conserved domain analysis of the encoded gene products (using the NCBI CD-search feature [https://www.ncbi.nlm.nil.gov/Structure/cdd/wrpsb.cgi]), prediction of transmembrane helices and membrane orientation (via the TMHMM Server [http://www.cbs.dtu.dk/services/TMHMM/]). These results were then compared against the results obtained for known phosphoglycerol or phosphoethanolamine transferases (LtaS from Staphylococcus aureus [c2w5tA], Lpt3 [NMB2010] and Ltp6 [NMA0408] from Neisseria meningitidis, EptB from E. coli [NC 000913.3], and Lpt6 from Haemophilus influenzae Rd [HI0275]), resulting in identification of four genes with common features. In Protein Homology/analogY Recognition Engine V2.0 (Phyre²; http://www.sbg.bio.ic.ac.uk/phyre2/html/page.cgi?id=index) for the gene product of the candidate cep2237, the top hit was t the phosphoglycerol transferase LtaS involved in lipoteichoic acid biosynthesis. Chromosomal deletion of cpe2237 was performed according t the method of Nariya et al (2011), and Western immunoblot analyses of whole cell lysates (as described in Example 4) revealed that the loss of cpe2237 corresponded to reduced reactivity with chicken anti-HN13 antiserum but enhanced reactivity with chicken anti-JGS4143 serum, Negative staining of wildtype and mutant lysates (according to the method of Castellanos-Serra and Hardy (2006)) confirmed that these results were not due to differences in the amount of immunogenic glycolipid produced between the wildtype and mutant, and complementation of the mutant wih a copy of the cpe2237 gene in trans (using pKRAH1) restored the reactivity against both antisera to wildtype levels.

It is postulated that the cpe2237 gene is the phosphoglycerol transferase, and that the immunogenic glycolipid in this mutant therefore lacks the PGro modifications. This results in the loss of signals corresponding to PGro in NMR analyses (eg. 1_{H}-13_{C} HSQC and/or TOCSY, 1_{H}-31_{P} HSQC) of both purified immunogenic glycolipid from the mutant (as described in Example #) and HR-MAS analysis of whole cells (as described by van Alphen et al (2014)). It is also anticipated that the loss of PGro will result in differential binding by human intelectin-1 (hItln1), which has been reported to recognize glycerol-phosphate groups on bacterial polysaccharide structures (Wesener et el (2015)). This is done either by performing a Western immunoblot blot on whole cell lysates in the same manner as in Example 3, using the hltln1 in lieu of a primary antibody and a fluorescently-labeled anti-hItln1 secondary antibody, or in microscopy of whole cells using fluorescently labeled hItln1. The loss of PGro correlating to reduced reactivity to the anti-HN13 antiserum indicates that PGro is an important epitope that contributes to the immune response to HN13, and supports the proposal that PGro is an important epitope in the elicitation of a broadly-crossreactive immune response by the immunodominant glycolipid.

The invention being thus described, it will be obvious that the same may be varied in many ways. Such variations are not to be regarded as a departure from the spirit and scope f the invention, and all such modifications as would be obvious to one skilled in the art are intended to be included within the scope of the following claims.

### Interpretation

References in the specification to “one embodiment”, “an embodiment”, etc., indicate that the embodiment described may include a particular aspect, feature, structure, or characteristic, but not every embodiment necessarily includes that aspect, feature, structure, or characteristic.

Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to affect or connect such module, aspect, feature, structure, r characteristic with other embodiments, whether or not explicitly described. In other words, any module, element or feature may be combined with any other element or feature in different embodiments, unless there is an obvious or inherent incompatibility, or it is specifically excluded.

It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an item, condition or step being referred to is an optional (not required) feature of the invention.

The singular forms “a,” “an,” and “the” include the plural reference unless the context clearly dictates otherwise. The term “and/or” means any one of the items, any combination of the items, or all of the items with which this term is associated. The phrase “one or more” is readily understood by one of skill in the art, particularly when read in context of its usage.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values and ranges proximate to the recited range that are equivalent in terms of the functionality of the composition, or the embodiment.

As will be understood by one skilled in the art, for any and all purposes, particularly in terms of providing a written description, all ranges recited herein also encompass any and all possible sub-ranges and combinations of sub-ranges thereat as well as the individual values making up the range, particularly integer values. A recited range includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc,

As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio.

### REFERENCES

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

1. Grass, J.E., Gould, L.H. & Mahon, B.E. Epidemiology of foodborne disease outbreaks caused by Clostridium perfringens, United States, 1998-2010. Foodborne pathogens and disease 10, 131-136 (2013).

2. Songer, J.G. Clostridial enteric diseases of domestic animals. Clin Microbiol Rev 9, 216-234 (1996).

3. Uzal, F.A., Vidal, J.E., McClane, B.A. & Gurjar, A.A. Clostridium Perfringens Toxins Involved in Mammalian Veterinary Diseases. Open Toxinology J 2, 24-42 (2010).

4. Al-Sheikhly, F. & Truscott, R.B. The interaction of Clostridium perfringens and its toxins in the production of necrotic enteritis of chickens. Avian diseases 21, 256-263 (1977a).

5. Timbermont, L. et al. Control of Clostridium perfringens-induced necrotic enteritis in broilers by target-released butyric acid, fatty acids and essential oils. Avian Pathol 39, 117-121 (2010).

6. Caly, D.L., R., Auclair, E. & Drider, D. Alternatives to Antibiotics to Prevent Necrotic Enteritis in Broiler Chickens: A Microbiologist's Perspective. Frontiers in microbiology 6, 1336 (2015).

7. Elwinger, K., Berndston, E., Engstrom, B., Fossum, O. & Waldenstedt, L. Effect of antibiotic growth promoters and anticoccidials on growth of Clostridium perfringens in the caeca and on performance of broiler chickens. Acta veterinaria Scandinavica 39, 433-441 (1998).

8. Hofacre, C.L., Beacom, T., Collett, S. & Mathis, G. Using competitive exclusion, mannan-oligosaccharide and other intestinal products to control necrotic enteritis. J Appl Poultry Res 12, 60-64 (2003).

9. .Hofshagen, M. & Kaldhusdal, M. Barley inclusion and avoparcin supplementation in broiler diets. 1. Effect on small intestinal bacterial flora and performance. Poultry science 71, 959-969 (1992).

10. Kaldhusdal, M., Schneitz, C., Hofshagen, M. & Skjerve, E. Reduced incidence of Clostridium perfringens-associated lesions and improved performance in broiler chickens treated with normal intestinal bacteria from adult fowl. Avian diseases 45, 149-156 (2001).

11. Van der Sluis, W. Clostridial enteritis is an often underestimated problem. World Poultry Journal 16, 43-43 (2000).

12. European-Union, Regulation (EC) No 1831/2003 of the European Parliament and of the Council. Official Journal of the European Union 46, 29-43 (2003).

13. Van Immerseel, F. et al. Clostridium perfringens in poultry: an emerging threat for animal and public health. Avian Pathol 33, 537-549 (2004).

14. Lovland, A. & Kaldhusdal, M. Severely impaired production performance in broiler flocks with high incidence of Clostridium perfringens-associated hepatitis. Avian Pathol 30, 73-81 (2001).

15. Hughes, J.A., Turnbull, P.C. & Stringer, M.F. A serotyping system for Clostridium welchii (C. perfringens) type A, and studies n the type-specific antigens. J Med Microbiol 9, 475-485 (1976).

16. Chemiak, R. & Frederick, H.M. Capsular polysaccharide of Clostridium perfringens Hobbs 9. Infection and immunity 15, 765-771 (1977).

17. Kalclkar, S., Glushka, J., van Halbeek, H., Morris, L.C. & Chemiak, R. Structure of the capsular polysaccharide of Clostridium perfringens Hobbs 5 as determined by NMR spectroscopy. Carbohydrate research 299, 119-128 (1997).

18. Sheng, S. & Chemiak, R. Structure of the capsular polysaccharide of Clostridium perfringens Hobbs 10 determined by NMR spectroscopy. Carbohydrate research 305, 65-72 (1997).

19. Richter, S.C. et al. Small molecule inhibitor of lipoteichoic acid synthesis is an antibiotic for Gram-positive bacteria. Proc Nati Acad Sci USA 110, 3531-3536 (2013).

20. Mot, D., Timbermont, L., Haesebrouck, F., Ducatelle, R. & Van Immerscel, F. Progress and problems in vaccination against necrotic enteritis in broiler chickens. Avian Pathol 43, 290-300 (2014).

21. Jiang, Y. et al. Protection Against Necrotic Enteritis in Broiler Chickens by Regulated Delayed Lysis Salmonella Vaccines. Avian diseases 59, 475-485 (2015).

22. Liu, H., Bouillaut, L., Sonenshein, A. L. & Melville, S. B. Use of a mariner-based transposon mutagenesis system to isolate Clostridium perfringens mutants deficient in gliding motility. Journal of bacteriology 195, 629-636 (2013).

23. Barbara, A.S., Trinh, H.T., Clock, R.D. & Glenn Songer, J. Necrotic enteritis-producing strains of Clostridium perfringens displace non-necrotic enteritis strains from the gut of chicks. Veterinary microbiology 126, 377-382 (2008).

24. Gohari, I. M., Parreira, V., Kropinski, A., Boerlin, P. & Prescott, J. F. Association of NetF-positive type A Clostridium perfringens with necrotizing enteritis in neonatal foals. Journal of Equine Veterinary Science 39, S23 (2016).

25. Nariya, Miyata, S., Suzuki, M., Tamai, E. & Okabe, A. Development and application of a method for counterselectable in-frame deletion in Clostridium perfringens. Applied and environmental microbiology 77, 1375-1382 (2011).

26. Chalmers, G. et al. Multilocus sequence typing analysis of Clostridium perfringens isolates from necrotic enteritis outbreaks in broiler chicken populations. Journal of clinical microbiology 46, 3957-3964 (2008).

27. Laemmli, U.K. Cleavage of structural proteins during the assembly of the head of bacteriophage T4. Nature 227, 680-685 (1970).

28. Burnette, W.N. “Western blotting”: electrophoretic transfer of proteins from sodium dodecyl sulfate-polyacrylamide gels to unmodified nitrocellulose and radiographic detection with antibody and radioiodinated protein A. Analytical biochemistry 112, 195-203 (1981).

29. Goyette-Desjardins, G. et al. Protection against Streptococcus suis Serotype 2 infection using a capsular polysaccharide glycoconjugate vaccine. Infect. immun. 84:2059-2075. (2016) 30. Orawan, C. and Acngwanich. Blood cell characteristics, hematological values and average daily gained weight of Thai indigenous, Thai indigenous crossbred and broiler chickens. Pakistan Journal of Biological Sciences. 10:302-309. (2007)

31. Westphal, 0., and K. Jann. Bacterial lipopolysaccharides: extraction with phenol-water and further applications of the procedure. Methods Carbohydr. Chem. 5:81-93. (1965)

32. Vinogradov, E., Aubry, A., and Logan, S.M. Structural characterization of wall and lipidated polysaccharides from Clostridium perfringens ATCC 13124, Carbohydr Res. 448:88-94 (2017).

33. Thompson DR Parreira VR, Kulkarni RR, Prescott JF. Live attenuated vaccine-based control of necrotic enteritis of broiler chickens, Vet Alicrobiol. 2006 Mar. 10;113(1-2):25-34. Epub 2005 Nov. 11.

1. An immunogenic glycan compound with a poly-β-1,4-ManNAc repeating-unit structure modified with at least one 6-linked phosphoglycerol.

2. The glycan of claim 1, which comprises a compound of Formula I in isolated, synthesized and/or purified form, optionally conjugated, or an immunogenic analogue or modified form thereof: 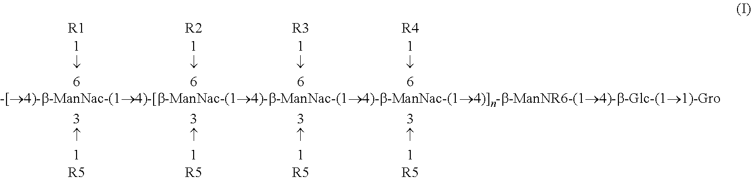
where n≥1; each of R1, R2, R3, and R4 comprises any modification, provided at least one of which is phosphoglycerol (—PGro); R5 comprises any modification; and R6 comprises —H or —Ac.

3. The glycan of claim 2 wherein R5 is OH and/or an R5 in a terminal copy of the repeating structure comprises a sugar, such as ribofuranose.

4. The glycan of claim 2 wherein at least one of R1-R4 is PGro and at least one, two or three of R1-R4 is phosphoethanolamine.

5. The glycan of any one of claims 2-4, wherein about 25% of R1-R4 is —PGro.

6. The glycan of claim 2 which comprises a compound of Formula II, or an immunogenic analogue or modified form thereof, where n≥1:

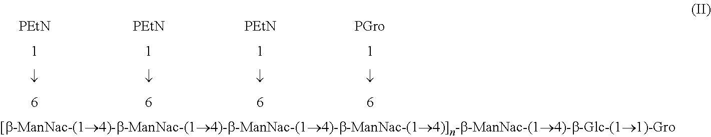

7. The glycan of claims 1, which is linked to a lipid, a single amino acid, an oligopeptide, a peptide, or a protein.

8. The glycan of any onc of claims 1-6, which is chemically conjugated to a biomolecule and expressed in a natural or heterologous host as an N-glycan, an O-glycan, on a lipid, on a cell surface, or on outer membrane vesicles (OMVs).

9. The glycan of claim 7, which is linked to a lipid, wherein the lipid is isolated and purified from a bacterial, archaeal or eukaryotic source, or is chemically synthesized.

10. The glycan of claim 9, wherein the lipid is linked to the glycan through a phosphate, a pyrophosphate linker or by a glycosidic linkage.

11. A vaccine comprising the glycan of claims 1, or an attenuated C. perfringens cell bearing the glycan or a bacteria engineered to heterologously express the glycan, and a pharmaceutically acceptable diluent, carrier, excipient, or adjuvant.

12. A method of treating or preventing an infection caused byC. perfringens by administrating a vaccine of claim 11.

13. A composition comprising an antibody or fragment thereof that specifically binds with a glycan of any one of claims 140, and a pharmaceutically acceptable diluent, carrier, or excipient.

14. A method of passively immunizing or treating an animal using the composition of claim 13.

15. A method of diagnosing an infection caused by a C. perfringens organism by using the composition of claim 13 to recognize C. perfringens species in a sample.

16. The method of claim 15 comprising the step of performing an immunoassay.
